(19) United States
(12) Patent Application Publication
(10) Pub. No.: US 2010/0016878 A1
(43) Pub. Date: Jan. 21, 2010

(54) TROCAR ASSEMBLY WITH OBTURATOR AND RETRACTABLE STYLET

(75) Inventor: Robert C. Smith, Middletown, CT (US)

Correspondence Address: Tyco Healthcare Group LP, 60 MIDDLETOWN AVENUE, NORTH HAVEN, CT, 06473, US

(73) Assignee: TYCO HEALTHCARE GROUP LP, North Haven, CT (US)

(21) Appl. No.: 12/513,439

(22) PCT Filed: Dec. 14, 2007
(86) PCT No.: PCT/US2007/025560

§ 371 Date: May 4, 2009

Related U.S. Application Data
(60) Provisional application No. 60/875,147, filed on Dec. 15, 2006.

(51) Int. Cl. A61B 17/34

(52) U.S. Cl. 606/185

(57) ABSTRACT

A surgical system for penetrating tissue includes a cannula having a cannula housing and a cannula sleeve extending from the cannula housing and an obturator at least partially positionable within the cannula. The obturator includes an obturator housing, an obturator member extending from the obturator housing and having a penetrating member adapted to penetrate tissue and a stylet at least partially disposed within the obturator member. The stylet and the obturator member are adapted for relative longitudinal movement between a first relative position corresponding to an unarmed condition of the penetrating member and a second relative position corresponding to an armed condition of the penetrating member.

### BACKGROUND

1. Technical Field

The present disclosure relates to a trocar assembly for use in minimally invasive surgical procedures, such as endoscopic or laparoscopic type procedures.

2. Background of the Related Art

Minimally invasive procedures are continually increasing in number and variation. Forming a relatively small diameter temporary pathway to the surgical site is a key feature of most minimally invasive surgical procedures. The most common method of providing such a pathway is by inserting a trocar assembly through the skin. In many procedures, the trocar assembly is inserted into an insufflated body cavity of a patient. In such procedures, the trocar assemblies with seal mechanisms are utilized to provide the necessary pathway to the surgical site while minimizing leakage of insufflation gases.

Trocar assemblies typically include an obturator which is removably inserted through a cannula. The obturator may include a safety shield which protects against unintentional puncturing by the sharpened tip of the obturator. The safety shield includes a mechanism which controls the relative movement and locking of the safety shield. One example of a safety shield mechanism is disclosed in commonly assigned U.S. Pat. No. 6,319,266 to Stellon et al., the entire contents of which are hereby incorporated by reference.

### SUMMARY

Accordingly, the present disclosure is directed to further improvements in trocar technology. In one preferred embodiment, a surgical system for penetrating tissue includes a cannula having a cannula housing and a cannula sleeve extending from the cannula housing, and an obturator at least partially positionable within the cannula. The obturator is adapted to pass through tissue, and is removable from the cannula subsequent to accessing an underlying tissue site. The obturator includes an obturator housing, an obturator member extending from the obturator housing and having a penetrating member adapted to penetrate tissue and a stylet at least partially disposed within the obturator member. The stylet and the obturator member are adapted for relative longitudinal movement between a first relative position corresponding to an unarmed condition of the penetrating member and a second relative position corresponding to an armed condition of the penetrating member. A latch member is associated with the obturator housing. The latch member is moveable from an initial position securing the stylet and the obturator in the first relative position to a release position permitting relative movement of the stylet and the obturator to the second relative position.

The stylet may be adapted for longitudinal movement relative to the obturator housing between an extended position corresponding to the unarmed condition of the penetrating end of the obturator member and a retracted position corresponding to the armed condition of the obturator member. The stylet is dimensioned to at least partially extend beyond the penetrating member of the obturator member when in the extended position. The stylet may be normally biased toward the extended position. The latch member may be adapted to secure the stylet in the extended position when in the initial position of the latch member and may be adapted to permit movement of the stylet to the retracted position when in the release position of the latch member.

A release member may be mounted to the obturator housing and operatively coupled with the latch member. The release member may be adapted to move the latch member to the release position during positioning of the obturator within the longitudinal opening of the cannula. The latch member may be monolithically formed with the obturator housing and arranged in cantilever relation therewith and being normally biased toward the initial portion thereof. Alternatively, the release member is adapted for movement relative to the obturator housing during positioning of the obturator within the longitudinal opening of the cannula, to thereby move the latch member to the release position. The release member may include a release button. The release button is dimensioned to extend distally beyond the obturator housing, and positioned to engage a cannula housing of the cannula upon mating of the obturator housing and the cannula housing whereby a generally proximally directed force applied by the cannula housing on the release button causes displacement of the release member and movement of the latch member to the release position.

The penetrating end may include a tapered portion extending towards a penetrating tip. The penetrating tip may define a beveled edge. The penetrating end may include a cylindrical portion disposed between the penetrating tip and the tapered portion.

### BRIEF DESCRIPTION OF THE DRAWINGS

Preferred embodiments of the present disclosure are described hereinbelow with references to the drawings, wherein:

FIG. 1 is a perspective view of a trocar assembly in accordance with the principles of the present disclosure;

FIG. 2 is a perspective view with parts separated of the obturator assembly illustrating the cannula assembly and the obturator assembly;

FIG. 3 is a perspective view with parts separated of the obturator assembly illustrating the obturator housing, the obturator member and the stylet;

FIG. 4 is a side cross-sectional view of the obturator assembly;

FIG. 5 is an enlarged cross-sectional view of the indicated area of detail of FIG. 4;

FIG. 6 is a second enlarged cross-sectional view of the obturator housing of the obturator assembly;

FIG. 7 is a perspective view of the indicator collar of the obturator housing;

FIG. 8 is a perspective view of the housing cover of the obturator housing;

FIG. 9 is a perspective view of the housing base of the obturator housing;

FIGS. 10-11 are perspective views illustrating the components of the latch mechanism;

FIG. 12 is a perspective view in partial cross-section taken along the lines 12-12 of FIG. 1 illustrating the obturator member and the stylet;

FIGS. 13-14 are perspective views illustrating the stylet in respective advanced and retracted positions relative to the obturator member;

FIG. 15 is a side cross-sectional view of the trocar assembly illustrating the obturator assembly mounted relative to the cannula assembly and the latch member in an actuated position;

FIG. 16 is a view of the indicated area of detail of FIG. 15 illustrating the relationship of the components of the latch member in the actuated position;

FIG. 17 is a view similar to the view of FIG. 15 illustrating the stylet of the obturator assembly in a retracted position;

FIG. 18 is a view of the indicated area of detail of FIG. 17 illustrating the relationship of the components of the latch member when the stylet is in the retracted position;

FIG. 19 is a perspective view illustrating insertion of the trocar assembly within tissue; and

FIG. 20 is a view similar to the view of FIG. 19 illustrating rotation of the obturator housing of the obturator assembly.

### DETAIL DESCRIPTION OF PREFERRED EMBODIMENTS

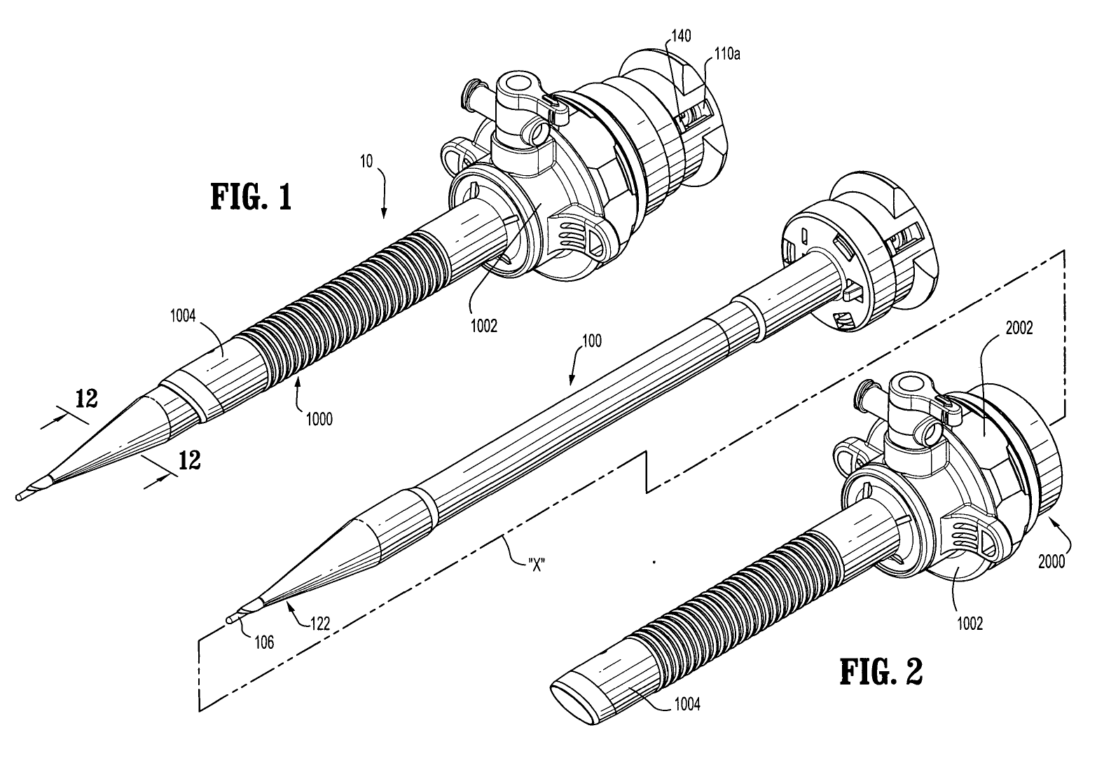

Referring now in detail to the drawing figures, in which, like references numerals identify similar or identical elements, there is illustrated, in FIGS. 1 and 2, a trocar assembly constructed in accordance with a preferred embodiment of the present disclosure and designated generally by reference numeral 10. Trocar assembly 10 is particularly adapted for use in minimally invasive surgical procedures such as endoscopic or laparoscopic procedures. Generally, trocar assembly 10 includes two principal subassemblies, namely, obturator assembly 100 and cannula assembly 1000.

Cannula assembly 1000 may be any cannula assembly suitable for use in a laparoscopic surgical procedure. In one preferred embodiment, cannula assembly 1000 includes cannula housing 1002 and cannula sleeve 1004 extending from the cannula housing 1002. Either or both cannula housing 1002 and cannula sleeve 1004 may be transparent in part or in whole and may be fabricated from biocompatible metal or polymeric material. Cannula assembly 1000 may include an internal seal such as a duck-bill valve or other zero closure valve adapted to close in the absence of a surgical instrument to prevent passage of insufflation gases through the cannula assembly 1000.

Trocar assembly 10 may also include a seal assembly 2000 which is preferably releasably mounted to cannula housing 1002. Means for releasably connected seal assembly 2000 to cannula housing 1002 may include a bayonet coupling, threaded connection, latch, friction fit, tongue and groove arrangements, snap-fit, etc. Seal assembly 2000 includes seal housing 2002 and at least one internal seal which is adapted to form a fluid tight seal about an instrument inserted through the seal assembly 2000. One suitable seal may be the fabric seal disclosed in commonly assigned U.S. Pat. No. 6,702,787, which issued Mar. 9, 2004, the entire contents of which are incorporated herein by reference. The seal disclosed in the '630 patent may be a flat septum seal having a first layer of resilient material and a second fabric layer juxtaposed relative to the first layer. Further details of the seal may be ascertained by reference to the ″787 patent. Seal assembly 2000 may or may not be a component of cannula assembly 1000. For example, the seal assembly may be a separate, removable assembly. In the alternative, the seal assembly may comprise an integral part of the cannula assembly 1000 and not be removable.

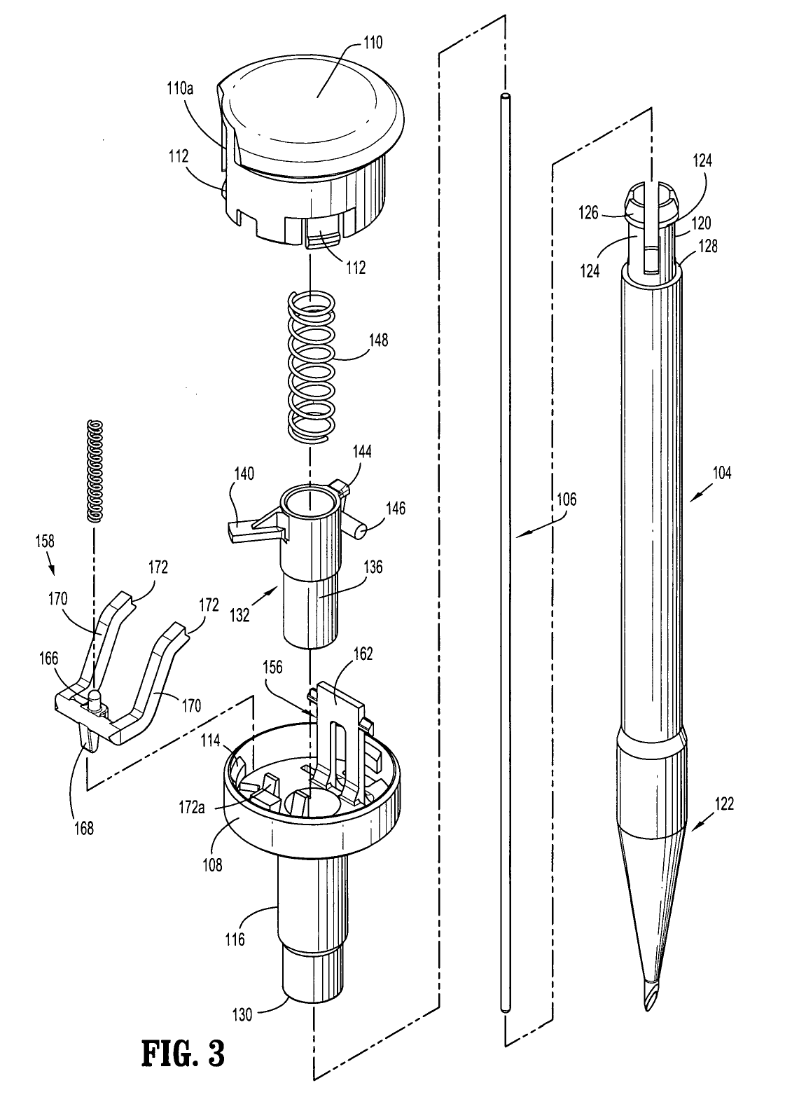

With reference now to FIGS. 3-5, in conjunction with FIG. 2, obturator assembly 100 includes obturator housing 102, elongated obturator member 104 extending distally from the housing 102 and stylet 106 at least partially disposed within the obturator member 104. Obturator member 104 defines obturator axis “x” and will be discussed in greater detail hereinbelow. Obturator housing 102 includes housing base 108 and housing cover 110. Once the appropriate components are positioned therewithin (as described below), housing base 108 may be attached to housing cover 110 by engaging mating surfaces, for example, by resilient latches 112 of cover 110 interlocking with correspondingly dimensioned latch openings 114 of housing base 108. Preferably, to uniformly connect base 108 and cover 110 at least three corresponding latches 112 and openings 114 are spaced evenly around the circumference of the cover 110 and the base 108, respectively. Housing base 108 further defines base extension 116. Base extension 116 includes a stepped configuration defining an internal shelf 118 as best depicted in FIG. 5. Base extension 116 receives obturator member 104 to mount the obturator member 104 to the obturator housing 102.

With reference to FIGS. 3-5, obturator member 104 includes mounting extension 120 at its proximal end and penetrating end 122 at its distal end. Penetrating end 122 will be discussed in greater detail hereinbelow. Mounting extension 120 includes a plurality, e.g., four, of axially depending tabs 124. Tabs 124 define circumferential ledges 126. Tabs 124 are adapted to move radially inwardly upon initial insertion of mounting extension 120 within base extension 116 of obturator base 108, and thereafter return to their initial outward positions after circumferential ledges 126 clear internal shelf 118. In such position, circumferential ledges 126 of mounting extension 120 securely engage internal shelf 118 of housing base 108. Mounting extension 120 also defines outer annular shelf 128 which contacts the distal end 130 of base extension 116 thereby preventing movement of obturator member 104 in a proximal direction. Thus, the aforedescribed mounting arrangement of obturator member 104 and housing base 108 secures the obturator member 104 from moving in an axial direction relative to obturator housing 102. However, obturator housing 102 may rotate relative to obturator member 104. In particular, circumferential ledges 126 of obturator member 104 are dimensioned to slide along internal shelf 118 of base extension 106 during manipulation and use of the obturator assembly 100. Thus, during insertion, the operator may rotate obturator housing 102 without concern of underlying rotation of penetrating end 122 of obturator member 104.

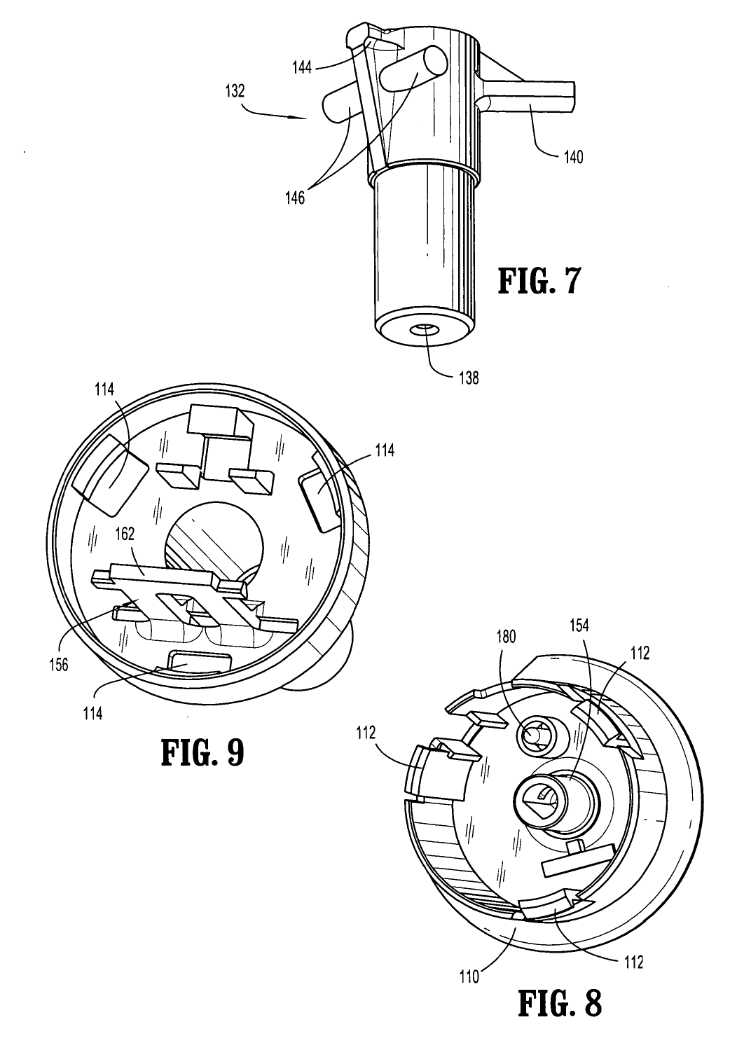

Referring now to FIGS. 5-7, in conjunction with FIG. 3, obturator assembly 100 further includes indicator collar 132 positioned within obturator housing 102 and secured to proximal end 134 of stylet 106. In one preferred arrangement, indicator collar 132 incorporates cylindrical section 136 defining internal bore 138 which receives proximal end 134 of stylet 106. Stylet 106 may be fixed within internal bore 138 through conventional means including cements, adhesives, interference fit, etc. With this arrangement, indicator collar 132 and stylet 106 move concurrently along the longitudinal axis “x” and relative to obturator housing 102 during use of the instrument.

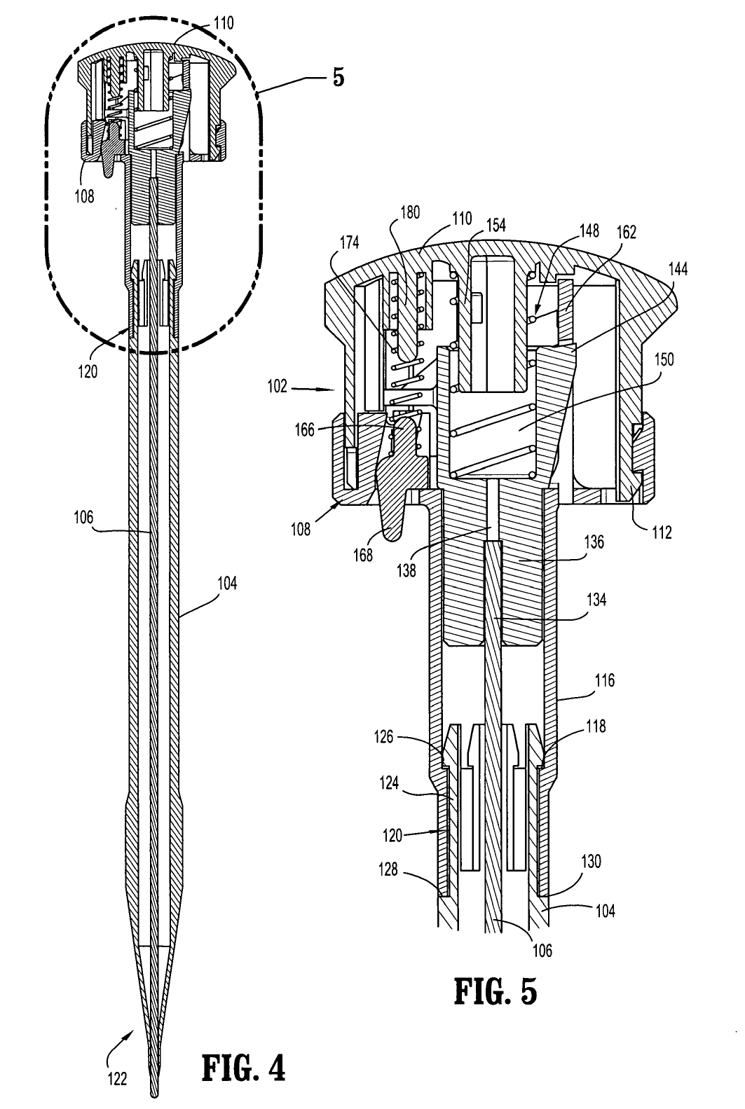

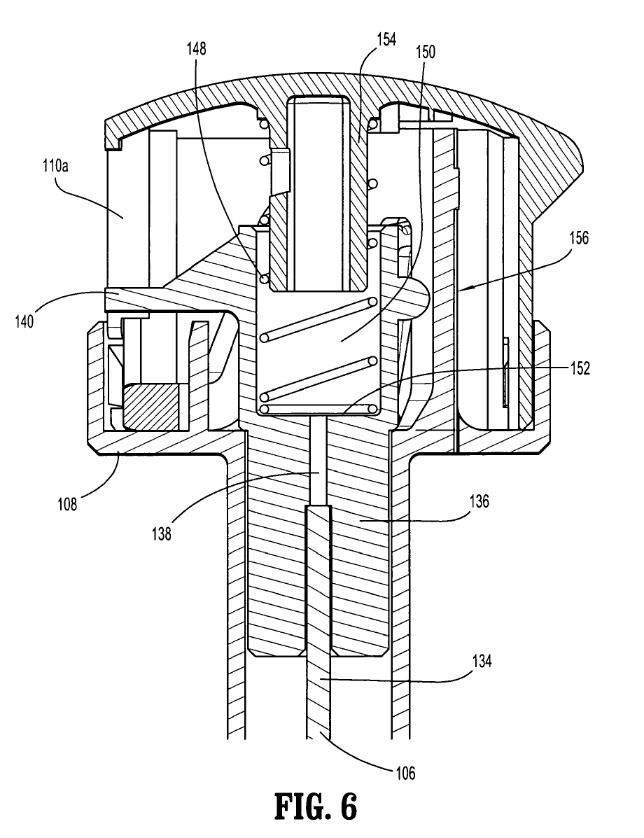

Indicator collar 132 further includes a shield position indicator, such as indicator flag 140, extending transversely relative to the indicator collar 132. Indicator flag 140 is visible from the exterior of obturator housing 102 as it extends through groove 110a of housing cover 110 (see also FIGS. 2 and 6). Preferably, indicator flag 140 is colored to contrast sharply with the surrounding housing components. For example, indicator flag 140 may be red if the surrounding housing components are white or light colored. Indicator collar 132 further includes collar ledge 144 and a pair of posts 146 formed below the ledge 144 and extending radially outwardly from the ledge 144. Collar ledge 144 serves to releasably lock stylet 106 in a distal position corresponding to an unarmed condition of obturator assembly 100. FIG. 4 illustrates the unarmed condition of obturator assembly 100.

Indicator flag 140 and stylet 106 are spring biased in the distal direction by coil spring 148. In particular, coil spring 148 is received within internal bore 150 of indicator collar 132 and engages internal shelf 152 (see FIG. 6) of the indicator collar 132. The proximal end of coil spring 138 is coaxially mounted about spring mount 154 (see also FIG. 8) depending from the interior surface of housing cover 110.

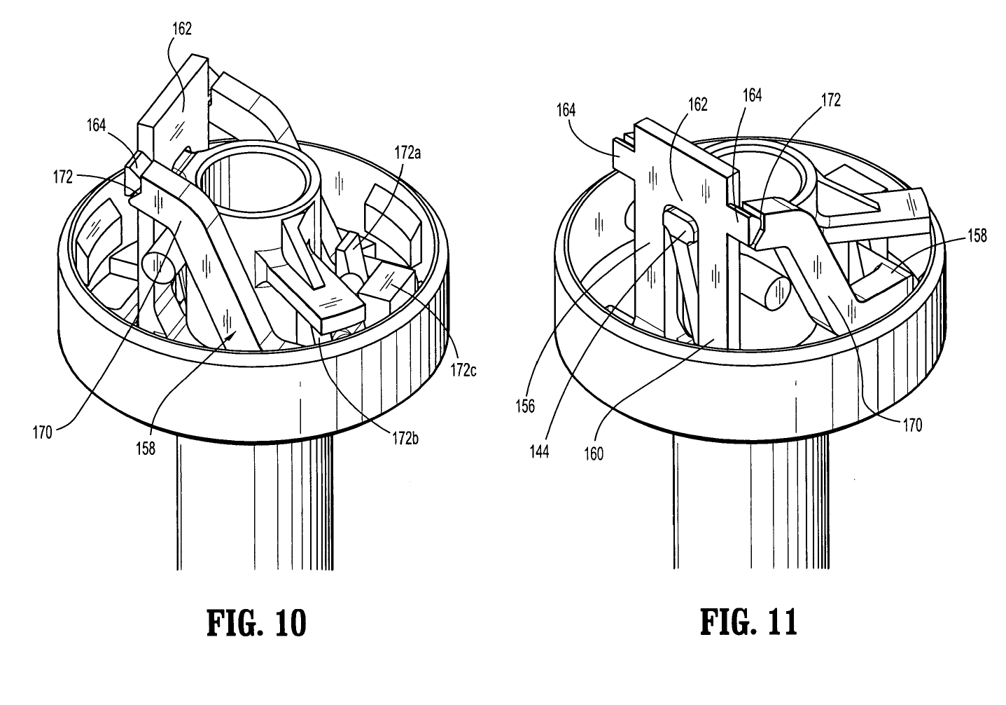

Referring now to FIGS. 9-11, in conjunction with FIGS. 3-5, obturator assembly 100 includes a latching mechanism disposed within obturator housing 102 to prevent proximal movement of stylet 106 until such time as the obturator assembly 100 is mounted to cannula assembly 1000 and the surgeon is prepared to begin trocar entry. Latching mechanism includes latch member 156, and release member such as slider 158, as best seen in FIG. 3. Latch member 156 has two vertical legs 160 connected by web 162. A pair of biasing posts 164 extends outwardly, one for each side of latch member 156. Collar ledge 144 of indicator collar 132 is engaged and secured by web 162 of latch member 156 when in an initial position of the latch member 156 as depicted in FIGS. 10-11. In the initial position of latch member 156, stylet 106 is retained in a first extended position shown in FIG. 4 corresponding to the unarmed condition of obturator assembly 100. Latch member 156 is preferably molded as part of housing base 106 in cantilever fashion. However, latch member 156 may be formed as a separate element and secured to base 106 by suitable known techniques.

Slider 158 includes post 166 disposed at its lower end, arming button 168 extending distally from the distal face of slider 158 and a pair of slider legs 170 which terminate in crooks 172. Crooks 172 defined in slider legs 170 are configured and dimensioned to engage posts 164 of latch member 156, as shown in FIGS. 10 and 11. Slider 158 is distally biased by slider spring 174 which is maintained in axial alignment by slider post 166 of slider 158. The proximal end of slider spring 174 bears against the inner surface of housing cover 110 and is maintained in position between proximal post 178 and cylindrical post 180 formed in the housing cover 110 (see FIG. 5). The distal biasing of slider 158 causes arming button 168 to project through opening 182 formed in housing base 106. The lower end or transverse leg 184 of slider 158 resides with mounting posts 172 a-c of housing base 106 with post base 184 of slider 158 residing within mounting posts 172b, 172c. (See FIG. 10). Mounting of obturator assembly 100 relative to cannula assembly 112 causes slider 158 to translate or rotate generally vertically in a generally proximal direction as will be described further hereinbelow.

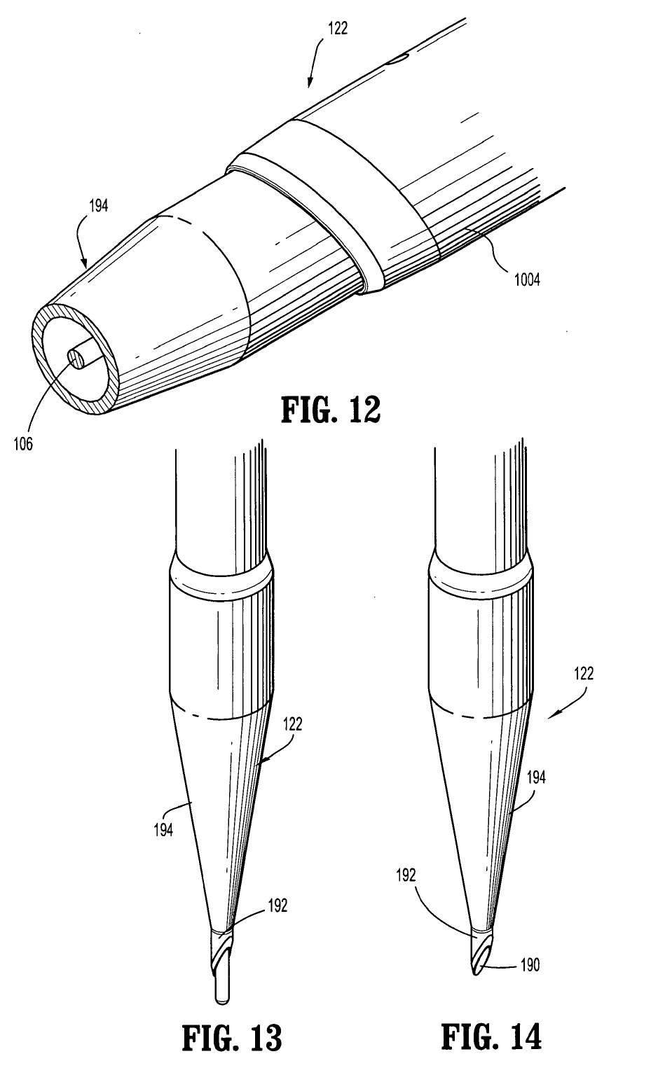

With reference to FIGS. 12-14, penetrating end 122 of obturator member 104 will be discussed. Penetrating end 122 of obturator member 104 generally tapers inwardly relative to longitudinal axis “x” towards penetrating tip 190. Penetrating tip 190 defines a general beveled arrangement, and may have a sharpened edge or may be generally blunt if desired. Extending contiguously from penetrating tip 190 is an annular, e.g., cylindrical, end portion 192 of relatively small diameter. Cylindrical end portion 192 provides a reduced profile when viewed axially to facilitate initial passage of penetrating end 122 through tissue. Tapered section 194 extends contiguously from cylindrical end portion 192.

Stylet 106 may be any suitable relatively rigid or slightly flexible member. Stylet 106 preferably defines a rounded or blunted end 106a to minimize trauma to tissue.

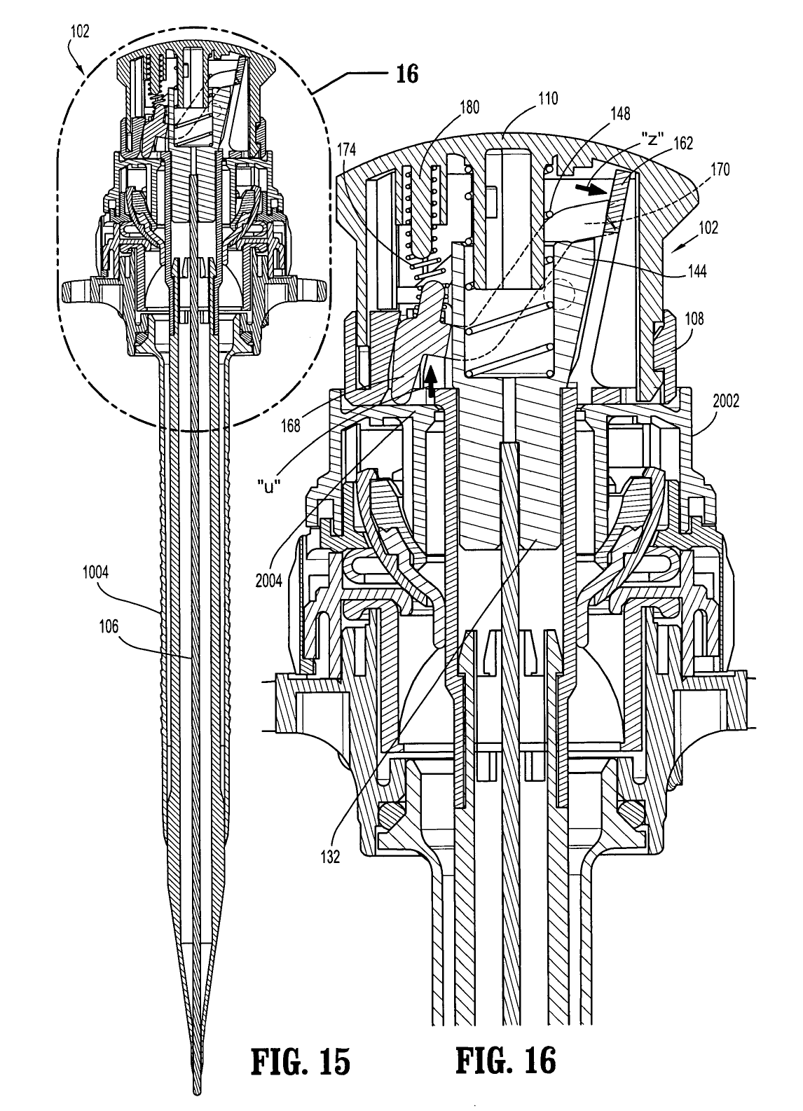

With reference now to FIGS. 15-16, a method of use and operation of trocar assembly 10 will be discussed. Obturator assembly 100 is inserted within cannula assembly 1000 and advanced to where obturator housing 102 is approximated with seal housing 2002 of the seal assembly 2000. Seal assembly 2000 may comprise a separate part or may be a component of cannula assembly 1000. Seal housing 2002 and housing base 108 of obturator housing 102 may be appropriately dimensioned to form a friction fit or may be coupled to each other by conventional means including bayonet coupling, tongue-groove, etc. Approximating the obturator housing 102 and the seal housing 202 releases the stylet 106 from a locked condition, actuating the trocar assembly. With the obturator housing 102 and seal housing 2002 approximated, arming button 168 of slider 158 engages surface 2004 of seal housing 2002 and is forced upwardly (depicted by directional arrow “u”) from the position depicted in FIG. 5 to the position depicted in FIGS. 15-16. During this movement, slider 158 pivots or angulates whereby legs 170 of slider 158 push latch member 156 in a radial outward direction (depicted by directional arrow “z”) such that web portion 162 of latch member 156 is out of axial alignment with ledge 144 of indicator collar 132. In this position, indicator collar 132 and stylet 106 are free to axially move.

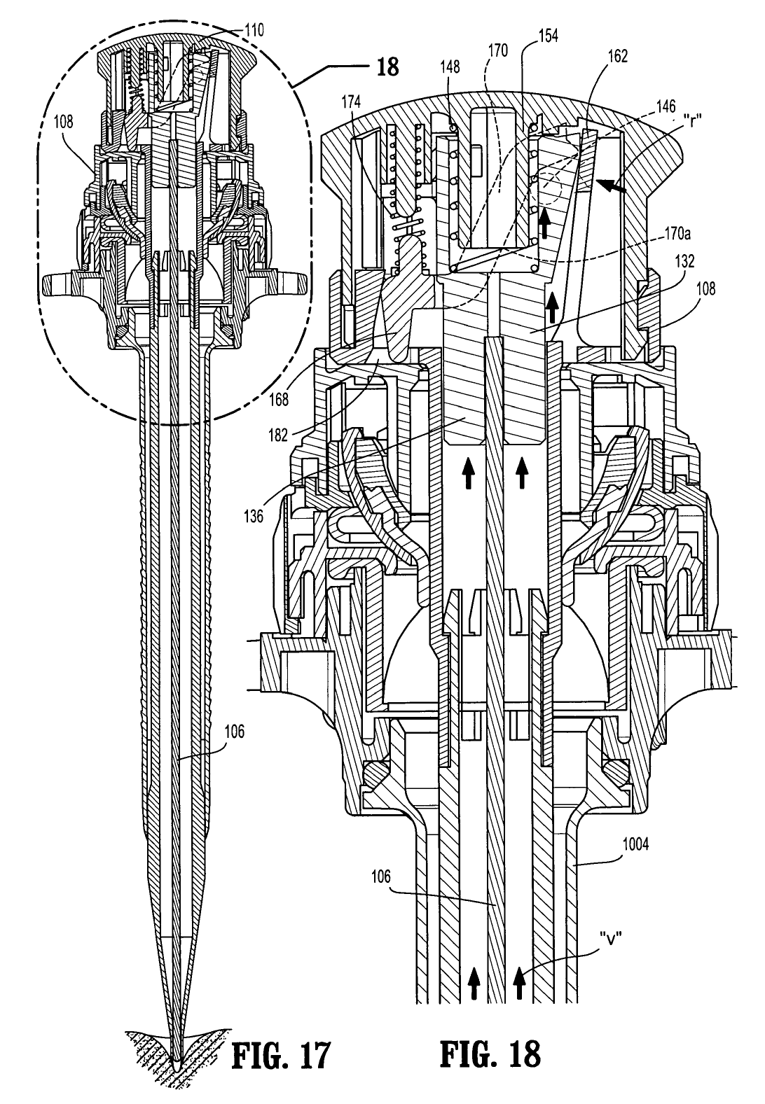

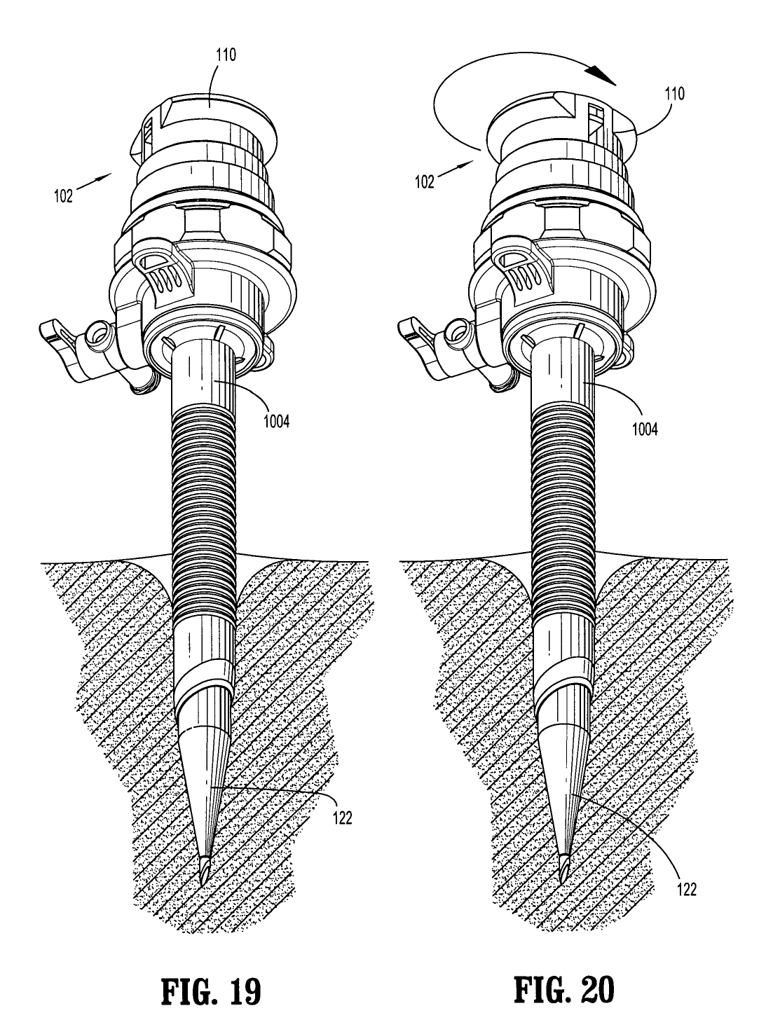

Referring now to FIGS. 17-18, the surgeon begins to insert trocar assembly 10 through the body wall of the patient. Stylet 106 contacts the tissue and is driven upwardly to cause the stylet 106 and indicator collar 132 to move proximally (depicted by directional arrow “v”) against the bias of coil spring 148. Such movement exposes the penetrating tip 190 of penetrating end 122 of obturator member 104 to incise the tissue as shown in FIG. 17. This armed condition of obturator assembly 100 is visually verified by the proximal location of indicator flag 140 of indicator collar 132. In addition, proximal movement of indicator collar 132 causes posts 146 of the indicator collar 132 to ride along outer surfaces 170a of legs 170 of slider 158 to thereby move the slider 158 at least radially inwardly and upwardly (as shown by the directional arrows “r”) in a general aligned position relative to the obturator axis “x”. FIG. 18 illustrates this actuated position of latch member 156. With penetrating tip 190 exposed, the surgeon may apply a distally-directed force to obturator assembly 100 to cause penetration through the tissue. The surgeon may rotate obturator housing 102 during manipulation and advancement of obturator assembly 100 into the tissue without concern of corresponding rotation of penetrating end 122. FIGS. 19-20 illustrate rotation of obturator housing 102 while penetrating end 122 remains stationary during advancement of trocar assembly 10 within tissue “t”. It is noted that indicator flag 140 in its proximal position provides visual confirmation of the armed condition of knife blade 190.

Once penetrating tip 190 and stylet 106 pass through the body wall of the patient, the stylet 106 moves distally to extend beyond penetrating tip 190 to assume the unarmed condition depicted in FIG. 4. In particular, indicator collar 132 and stylet 106 are driven distally under the influence of coil spring 148. Concurrently with this movement, slider 158, which is aligned relative to axis “x”, is driven distally under the influence of coil spring 174. In the respective positions of indicator collar 132 and slider 158 depicted in FIGS. 4 and 5, collar ledge 144 of indicator collar 132 securely engages web 162 of latch member 156 to secure stylet 106 in the extended position. The obturator assembly 100 is removed from cannula assembly 1000 and surgery is performed with instruments inserted through cannula assembly 1000.

The materials utilized in the components of the presently disclosed trocar assembly generally include materials such as, for example, ABS, polycarbonate, stainless steel, titanium and any other suitable biocompatible metals and/or polymeric materials. A preferred ABS material is CYCOLAC which is available from General Electric. A preferred polycarbonate material is also available from General Electric under the trademark LEXAN. An alternative polycarbonate material which may be utilized is CALIBRE polycarbonate available from Dow Chemical Company. The polycarbonate materials may be partially glass filled for added strength.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure.

What is claimed is:

1. A surgical system for penetrating tissue, which comprises:
a cannula including a cannula housing and a cannula sleeve extending from the cannula housing;
an obturator at least partially positionable within the cannula and being adapted to pass through tissue, the obturator being removable from the cannula subsequent to accessing an underlying tissue site, the obturator including:
an obturator housing;
an obturator member extending from the obturator housing and having a penetrating member adapted to penetrate tissue;
a stylet at least partially disposed within the obturator member, the stylet and the obturator member being adapted for relative longitudinal movement between a first relative position corresponding to an unarmed condition of the penetrating member and a second relative position corresponding to an armed condition of the penetrating member; and
a latch member associated with the obturator housing, the latch member being moveable from an initial position securing the stylet and the obturator in the first relative position to a release position permitting relative movement of the stylet and the obturator to the second relative position.

2. The surgical system according to claim 1 wherein the stylet is adapted for longitudinal movement relative to the obturator housing between an extended position corresponding to the unarmed condition of the penetrating end of the obturator member and a retracted position corresponding to the armed condition of the obturator member.

3. The surgical system according to claim 2 wherein the stylet is dimensioned to at least partially extend beyond the penetrating member of the obturator member when in the extended position.

4. The surgical system according to claim 3 wherein the stylet is normally biased toward the extended position.

5. The surgical system according to claim 4 including wherein the latch member is adapted to secure the stylet in the extended position when in the initial position of the latch member and is adapted to permit movement of the stylet to the retracted position when in the release position of the latch member.

6. The surgical system according to claim 5 including a release member mounted to the obturator housing and operatively coupled with the latch member, the release member adapted to move the latch member to the release position during positioning of the obturator within the longitudinal opening of the cannula.

7. The surgical system according to claim 6 wherein the latch member is monolithically formed with the obturator housing and arranged in cantilever relation therewith and being normally biased toward the initial portion thereof.

8. The surgical system according to claim 6 wherein the release member is adapted for movement relative to the obturator housing during positioning of the obturator within the longitudinal opening of the cannula, to thereby move the latch member to the release position.

9. The surgical system according to claim 8 wherein the release member includes a release button, the release button dimensioned to extend distally beyond the obturator housing, and positioned to engage a cannula housing of the cannula upon mating of the obturator housing and the cannula housing whereby a generally proximally directed force applied by the cannula housing on the release button causes displacement of the release member and movement of the latch member to the release position.

10. The surgical system according to claim 1 wherein the penetrating end includes a tapered portion extending towards a penetrating tip.

11. The surgical system according to claim 10 wherein the penetrating tip defines a beveled edge.

12. The surgical system according to claim 10 wherein the penetrating end includes a cylindrical portion disposed between the penetrating tip and the tapered portion.
